(19) United States
(12) Patent Application Publication
(10) Pub. No.: US 2008/0069863 A1
(43) Pub. Date: Mar. 20, 2008

(54) Device for treatment of disorders in the oral cavity with nitric oxide, and manufacturing process for the same

(76) Inventor: Tor Peters, Helsingborg (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/891,674

(22) Filed: Aug. 10, 2007

Related U.S. Application Data
(63) Continuation of application No. PCT/EP06/50888, filed on Feb. 13, 2006.
(60) Provisional application No. 60/652,758, filed on Feb. 14, 2005.

(30) Foreign Application Priority Data
Feb. 11, 2005 (EP) 05002937.0

(51) Int. Cl. A61K 9/70; A61K 33/00; A61P 35/00; A61P 43/00; A61K 9/14

(52) U.S. Cl. 424/443; 424/489; 424/718

(57) ABSTRACT

A device and method for therapeutical treatment of disorders in the oral cavity and a process for manufacturing of said device is disclosed. The device comprises a nitric oxide (NO) eluting polymer. The nitric oxide (NO) eluting polymer is configured to elute a therapeutic dosage of nitric oxide (NO) when used in the oral cavity. The device allows for target treatment of infections or wounds in the oral cavity. The device comprising the nitric oxide (NO) eluting polymer is arranged to contact an infected area in the oral cavity, such that a therapeutic dose of nitric oxide is eluted from said nitric oxide eluting polymer to said area. The nitric oxide (NO) eluting polymer is integrated with a carrier material, such that said carrier material, in use, regulates and controls the elution of said therapeutic dosage of nitric oxide (NO).

### RELATED APPLICATIONS

This application is a continuation of PCT/EP2006/050888, filed Feb. 13, 2006, which claims priority to European Patent Application No. 05002937.0, filed Feb. 11, 2005; and U.S. Provisional Application No. 60/652,758, filed Feb. 14, 2005. The entire content of these applications is incorporated herein by reference.

### BACKGROUND OF THE INVENTION

1. Field of the Invention

This invention pertains in general to the field of therapeutical treatment of disorders in the oral cavity. More particularly the invention relates to a device and method of treatment of disorders in the oral cavity and a process for manufacturing said device, involving Nitric Oxide (NO).

2. Description of the Related Art

Infections by bacteria, viruses, fungi or yeasts are the underlying cause of many complications during wound care. A wide range of treatments has been developed to control such disorders, including physical and chemical methods and antimicrobial agents of a wide variety of antimicrobial agents. Despite the widespread use of these approaches, it is generally recognized that our ability to halt the invasion, persistence and spread of microbial infections remain limited.

Treatment of disorders in the oral cavity, such as paradontosis, is especially difficult, since the mucous membrane in the oral cavity is exposed to a variety of substances from the external (outside of the body) environment. Up to now the only reliable treatment of infections in the oral cavity appears to be the use of antibiotics. Treatment with antibiotics has certain disadvantages, such as that the bacteria develops tolerance and resistance to the antibiotics over time, and thus become difficult to eradicate.

It is known that nitric oxide (NO) provides an alternative to conventional therapies, such as antibiotics. Nitric oxide is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc., and general microbial invasion. This improvement of healing is partly caused by NO inhibiting the activation or aggregation of blood platelets, and also by NO causing a reduction of inflammatory processes at the site of an implant. Furthermore, NO has a vasodilating effect, which also affects, and promotes, the healing process.

NO is also known to have an anti-pathogenic, especially an anti-viral, effect, an anti-sacral effect, and furthermore NO has an anti-cancerous effect, as it is cytotoxic and cytostatic in therapeutic concentrations, i.e. it has among other effects tumoricidal and bacteriocidal effects. NO has for instance cytotoxic effects on human hematological malignant cells from patients with leukemia or lymphoma, whereby NO may be used as a chemotherapeutic agent for treating such hematological disorders, even when the cells have become resistant to conventional anti-cancer drugs.

However, due to the short half-life of NO, it has hitherto been very hard to treat viral, bacteria, virus, fungi or yeast infections with NO. This is because NO is actually toxic and has negative effects when applied in too large amounts to the body. NO is actually also a vasodilator, and too large amounts of NO introduced into the body will cause a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible.

US 2004/265244 discloses a composition and a method directed to antimicrobial release of NO, in order to prevent gingival and other mucosal diseases. The elution of NO from the device in US 2004/265244 is initiated by light activation of a nitosyl-containing organometallic compound. Hence, the activation process of US 2004/265244 is complicated. Furthermore, nothing is mentioned in US 2004/265244 about regulating the release of nitric oxide from the device.

U.S. Pat. No. 5,958,427 describes NO-donor compounds and pharmaceutical compositions containing such NO-donor compounds, for delivering NO to the apical surface of a mucosa. Nothing is mentioned in U.S. Pat. No. 5,958,427 about regulating the release of nitric oxide from the device.

EP 1 300 424 discloses extremely hydrophobic NO releasing polymers. These polymers are extensively cross-linked polyamine-derivatized divinylbenzene diazeniumdiolates. Since the polymer according to EP 1 300 424 is extremely hydrophobic, it is very unlikely that a sufficient elution on nitric oxide may be obtained in the oral cavity. The mentioning of polyethylenimine, page 9, line 35, is only in respect of excipient polymers to be included in blends and copolymers. Nothing is mentioned in EP 1 300 424 about regulating the release of nitric oxide from the device.

U.S. Pat. No. 5,691,423 discloses a polymeric, and pharmaceutical, composition capable of releasing NO, said polymeric composition comprising a polysaccharide including a NO releasing N₂O₂⁻ functional group bound to the polymer. Nothing is mentioned in U.S. Pat. No. 5,691,423 about regulating the release of nitric oxide from the device.

U.S. Pat. No. 6,737,447 discloses a coating for medical devices, which coating provides NO delivery by using nanofibers of L-PEI. U.S. Pat. No. 6,737,447 points out, and stresses, that the coating is insoluble in water. This can only be interpreted as the release of NO is initiated by something else than water. Furthermore, nothing is mentioned in U.S. Pat. No. 6,737,447 about regulating the release of nitric oxide from the device.

Other example for NO eluting polymers are given in U.S. Pat. No. 5,770,645, wherein polymers derivatized with at least one —NO_{X} group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of medical devices such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner to tissues and organs to aid the healing process and to prevent injury to tissues at risk of injury. Electrospun nanofibers of linear poly(ethylenimine) diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device.

However, the disclosure is both silent concerning an improvement of present technology in respect of treatment of disorders in the oral cavity, and the anti pathogenic potential of nitric oxide, and how such treatment could be regulated.

Hence, an improved, and more advantageous, device for the treatment and/or prevention of infection, caused by bacteria, viruses, fungi or yeast or herpes is desired. It is also desired that said device does not develop bacterial resistance, increases circulation, and acts as a healing promoter. It is further desired that the treatment could be regulated. It would be advantageous, in particular, to provide a device allowing for target treatment of both osteosynthetic and soft tissue healing post dental implant. Prevention and treatment of paradontosis or other infected wounds and cancer, and increased circulation, in the oral cavity, by means of such a device, would be advantageous.

### SUMMARY OF THE INVENTION

Accordingly, the present invention preferably seeks to mitigate, alleviate or eliminate one or more of the above-identified deficiencies in the art and disadvantages singly or in any combination and solves, among others, the problems mentioned above, by providing a device according to the appended patent claims.

According to one aspect of the invention, a device is provided that allows for target treatment of infections or wounds in the oral cavity. The device comprises a nitric oxide (NO) eluting polymer arranged to contact the infected area in the oral cavity, such that a therapeutic dose of nitric oxide is eluted from said nitric oxide eluting polymer to said area.

According to another aspect of the invention, a manufacturing process for such a device is provided, wherein the process is a process for forming a device that allows for target treatment of infections or wounds in the oral cavity. The process comprises selecting a plurality of nitric oxide eluting polymeric fibers, and deploying said nitric oxide eluting fibers in a patch or pad to be comprised in said device.

The present invention has at least the advantage over the prior art that it provides target exposure of an infected or wounded area to NO, whereby a very effective anti-viral, anti-bacterial, anti-fungi and/or anti-cancer therapy is achievable.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a sponge according to the invention,

FIG. 2 is a schematic illustration of a patch or pad according to the invention,

FIG. 3 is a schematic illustration of another patch or pad according to the invention, with NO-elution in one direction only,

FIG. 4 is a schematic illustration of a condom/sheath according to the invention,

FIG. 5 is a schematic illustration of nanoparticles, or microspheres, according to the invention,

FIG. 6 is a schematic illustration of a mouth wash according to the invention, and

FIG. 7 is an illustration of two elution profiles for two different mixtures of nitric oxide eluting polymer and carrier material.

### DETAILED DESCRIPTION OF THE PREFERRED EMBODIMENTS

The following description focuses on an embodiment of the present invention applicable to a device, in the form of a pad or patch, which allows for target treatment of infections or wounds in the oral cavity, such as paradontosis, herpes etc. However, also alternative embodiments are described.

With regard to nitric oxide (nitrogen monoxide, NO), its physiological and pharmacological roles have attracted much attention and thus have been studied. NO is synthesized from arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is 2 to 3 orders higher, and that iNOS produces an extremely large amount of NO.

In the case of the generation of a large amount of NO as in the case of the production by iNOS, it is known that NO reacts with active oxygen to attack exogenous microorganisms and cancer cells, but also to cause inflammation and tissue injury. On the other hand, in the case of the generation of a small amount of NO as in the case of the production by cNOS, it is considered that NO takes charge of various protective actions for a living body through cyclic GMP (cGMP), such as vasodilator action, improvement of the blood circulation, antiplatelet-aggregating action, antibacterial action, anticancer action, acceleration of the absorption at the digestive tract, renal function regulation, neurotransmitting action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible.

The polymers may be manufactured by electro spinning, gas spinning, air spinning, wet spinning, dry spinning, melt spinning, and gel spinning. Electro spinning is a process by which a suspended polymer is charged. At a characteristic voltage a fine jet of polymer releases from the surface in response to the tensile forces generated by interaction by an applied electric field with the electrical charge carried by the jet. This process produces a bundle of polymer fibers, such as nanofibers. This jet of polymer fibers may be directed to a surface to be treated.

In other embodiments the polymers may be manufactured by air spinning, wet spinning, dry spinning, melt spinning, or gel spinning.

Furthermore, U.S. Pat. No. 6,382,526, U.S. Pat. No. 6,520,425, and U.S. Pat. No. 6,695,992 disclose alternative processes and apparatuses for the production of such polymeric fibers. These techniques are generally based on gas stream spinning, also known within the fiber forming industry as air spinning, of liquids and/or solutions capable of forming fibers.

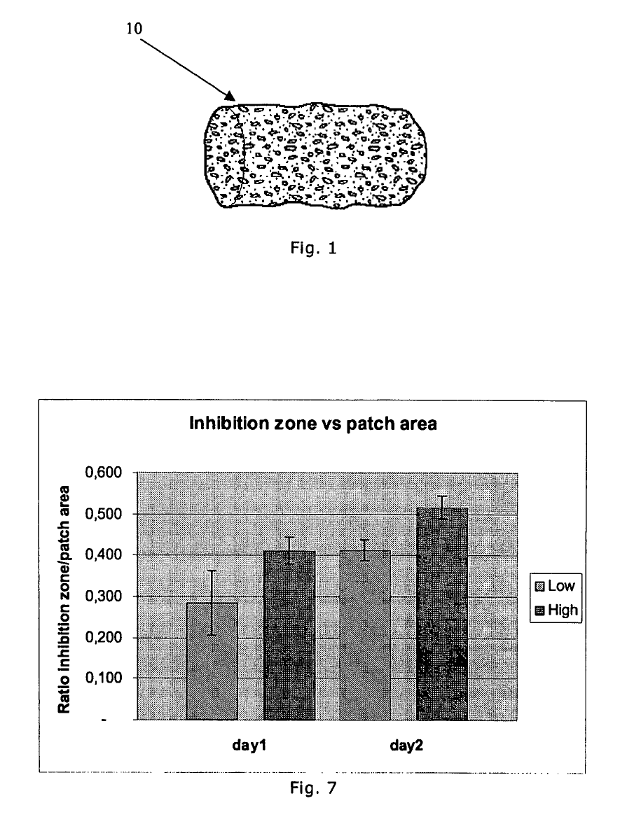

In an embodiment of the invention, according to FIG. 1, the device 10 is in form of a nano-spun, NO-eluting sponge or fiber coated sponge or sponge-like device, such as a sponge or a cotton ball or pillow. This NO-eluting sponge may be placed between the lip and teeth to increase circulation and prevent infection. When the nano-spun, NO-eluting sponge according to the present invention gets in contact with the moisture in the oral cavity the NO-eluting sponge starts to release NO to the area to be treated. This nano-spun NO-eluting sponge is preferably comprised of nano-spun fibers of a polymer that elutes NO. Such polymers are for example polyalkyleneimines, such as L-PEI (Linear PolyEthyleneImine) or B-PEI (Branched PolyEthyleneImine), which polymers have the advantage of being biodegradable to natural products or biocompatible with the latter.

This sponge has the advantage that it is easily activated. Furthermore, the NO is applied locally, without influencing other parts of the body, due to the short half-life of the NO eluted from the NO eluting polymer material. Thus, implications concerning the vascular system are kept very local and low, while at the same time the effect of NO is optimally exploited.

In another embodiment the sponge according to an embodiment of the present invention is applied to the oral cavity with the aid of a stick or pin at the area to be treated. This area may be anywhere in the oral cavity, such as between the gum and the teeth, between the teeth, in a dental pocket, etc. More specifically, the sponge is for instance releasably attached to the stick, preferably to an end of the stick. The end is then introduced into the oral cavity together with the sponge, where it is released from the stick, e.g. by counter holding the sponge with two fingers and drawing back the stick. The stick is thus removed from the oral cavity, leaving the sponge behind in the oral cavity for treatment therein.

Akron University has developed NO-eluting L-PEI molecule that can be nano-spun onto the surface of medical devices permanently implantable into the human body, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner.

However, the meaning of “controlled” in the context of U.S. Pat. No. 6,737,447 is only directed to the fact that nitric oxide is eluted from the coating during a period of time, i.e. that the nitric oxide is not eluted all in once. Therefore, the interpretation of “controlled” in respect of U.S. Pat. No. 6,737,447 is different from the meaning of “regulating” in the present invention. “Regulate or control”, according to the present invention is intended to be interpreted as the possibility to vary the elution of nitric oxide to thereby achieve different elution profiles.

A polymer comprising an O-nitrosylated group is also a possible nitric oxide eluting polymer. Thus, in one embodiment of the present invention, the nitric oxide eluting polymer comprises diazeniumdiolate groups, S-nitrosylated and O-nitrosylated groups, or any combinations thereof.

In still another embodiment of the present invention said nitric oxide eluting polymer is a poly(alkyleneimine)diazeniumdiolate, such as L-PEI-NO (linear poly(ethyleneimine)diazeniumdiolate), where said nitric oxide eluting polymer is loaded with nitric oxide through the diazeniumdiolate groups and arranged to release nitric oxide at a treatment site.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

In still another embodiment of the present invention the nitric oxide eluting polymer may be a O-derivatized NONOate. This kind of polymer often needs an enzymatic reaction to release nitric oxide.

Other ways of describing polymers, which may be suitable as nitric oxide eluting polymer, is polymers comprising secondary amine groups (═N—H), such as L-PEI, or have a secondary amine (═N—H) as a pendant, such as aminocellulose.

It is preferable that the nano-spun fibers in the NO-eluting sponge according to the embodiment of present invention comprise L-PEI. This embodiment of course permits the sponge to be placed in another location in the oral cavity than between the lip and the teeth. When placed on an area to be treated the device provides for promotion of osteosynthetic and soft tissue healing, as well as prevention and treatment of paradontosis, infections or wounds, thanks to the effect of NO eluting from the sponge into the regions to be treated. One of the advantages of electrospun or gas-jet spun nanofibers is their large surface area per volume unit. For the sponge this leads to a very effective treatment with a compact device.

One field of application of the device is post dental implant, e.g. accelerated healing thereof, or post-operative infection control, which is simplified and made more effective and convenient by the invention.

Other examples of NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups. Such polymers may also be used for other embodiments of the devices according to the present invention. However, L-PEI is preferred, as the NO is eluted without any secondary products that could lead to undesired side effects as a result of treatment with the devices described herein.

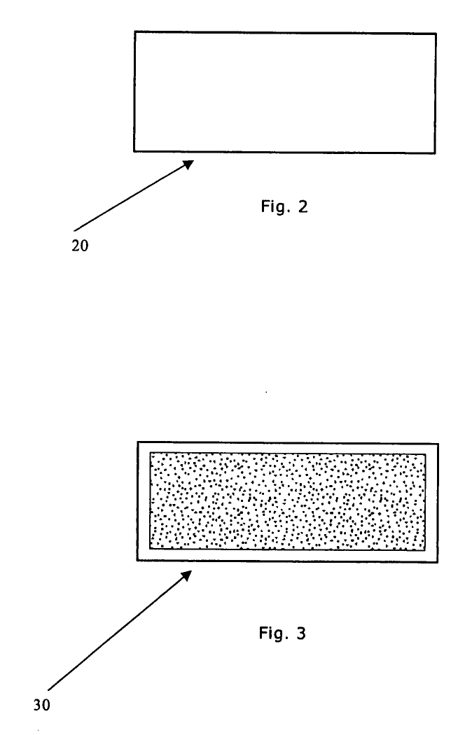

In another embodiment the device according to the present invention is in the form of a pad or patch 20, according to FIG. 2. The pad or patch is coated with or comprises at least partly, at least on one side, nano-spun fibers, which according to embodiments of the present invention comprise the materials mentioned above, regarding the sponge. The nano-spun fibers elute NO in a therapeutic dose as the nano-spun fibers that release NO, which is eluted from the fibers without harmful secondary or waste products, are activated for this purpose when they get in contact with the moisture in the oral cavity. This embodiment has the advantage that it is easily applicable, and removable, on, and from, the target area. Furthermore, this pad or patch has the advantage that it is easily activated. Furthermore, the NO is applied locally, without influencing other parts of the body, due to the short half-life of the NO eluted from the NO eluting polymer material. Alternatively, if the oral cavity for some reason should be deprived of humidity, the patch, and also other devices according to embodiments of the invention, may be activated immediately prior to introduction or in the cavity, e.g. by moisturizing them in a bath of water or by a water sprayed onto the device.

The device according to this embodiment of the present invention may in a further embodiment be soluble in the oral cavity. When the device is subjected to the moisture in the oral cavity, the device is disintegrated in its entirety, wherein the time for dissolving the device is adapted to specific requirements, as for instance therapeutic concentrations to be released over time. This embodiment has the advantage that it is easily applicable and does not have to be removed for replacement by another device or after the therapeutic treatment with the device is completed.

In another embodiment of the present invention the device or system only allows NO-elution in one direction. In this kind of embodiment one side of the device according to the invention has low permeability, or substantially no permeability, to nitric oxide. This may be accomplished by applying a material on one side of the device according to the invention that is not permeable to NO. Such materials may be chosen from the group comprising common plastics, such as fluoropolymers, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. This embodiment is also easy to manufacture as the NO eluting polymer, e.g. L-PEI (or nitric oxide eluting polymer and carrier material, which will be explained in more detail below) may be electro or gas-jet spun onto the surface of the device according to the invention of e.g. the mentioned plastics, latex, or cotton.

In still another embodiment the device is provided with one membrane, which is permeable to nitric oxide, on a first side of the device, and another membrane, which has low permeability or substantially no permeability to nitric oxide, on a second side of said device. This embodiment provides the possibility to direct the elution to said first of the device, while the elution of nitric oxide is substantially prevented from said second side. Thereby, a greater amount of nitric oxide will reach the intended area to be treated.

In another embodiment of the present invention the device only allows NO-elution in one direction, according to FIG. 3. In this kind of embodiment one side of the patch or pad 30 is non-permeable to NO. This may be accomplished by applying a material on one side of the patch or pad that is not permeable to NO. Such materials may be chosen from the group comprising common plastics, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. In this way, the therapeutic effect of NO is easily directable to certain regions in the oral cavity without interfering with other regions therein.

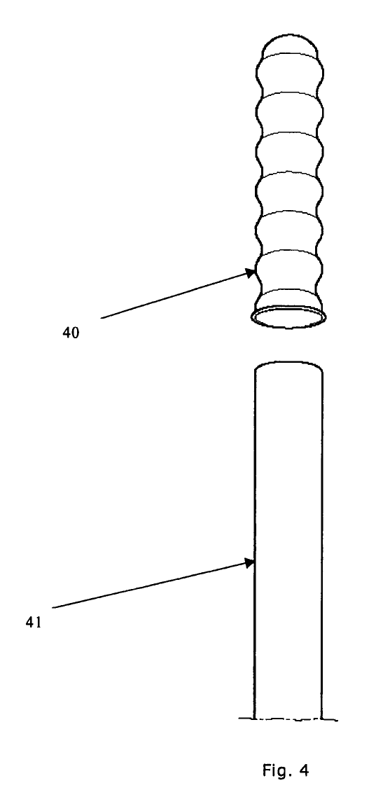

A further embodiment of the invention is illustrated in FIG. 4, in which the device is shaped as a condom/sheath 40, which either is made of an NO eluting polymer or coated with it, e.g. by nano electro-spinning or gas-jet spinning. According to this embodiment the condom/sheath may be mounted on a stick 41 for easier application. The condom/sheath may be applied by inserting the stick, with the condom/sheath according to the present invention mounted thereon, adjacent to the area to be treated. Then the stick may be extracted from the oral cavity. The condom/sheath is left adjacent to the area to be treated. This area may be areas such as in between the teeth, the tooth pocket, or any other area in the oral cavity where the condom/sheath is applicable.

This condom/sheath may of course be in any suitable size, such as a size suitable for inserting said condom/sheath between the teeth or in a tooth pocket. The condom/sheath is then inserted on the preferred area to be treated with the aid of a suitable means, such as a stick or pin. This embodiment has the advantages that it is easy to pin point the treatment area and it is easy to apply.

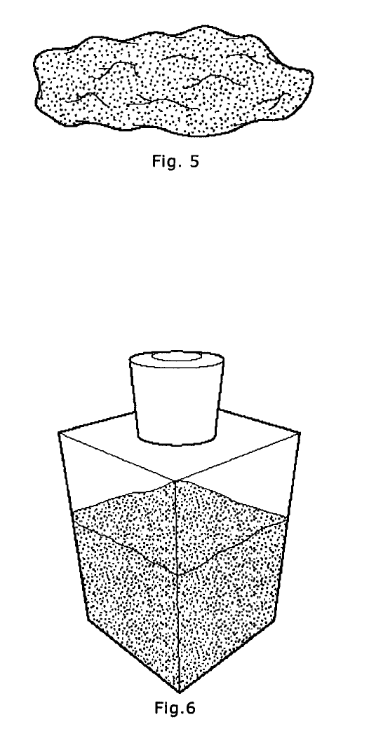

In still another embodiment of the present invention NO-eluting nanoparticles, or microspheres, may be formed from NO-eluting polymers, according to FIG. 5. These nanoparticles, e.g. in the form of microspheres may be integrated in a soluble film that disintegrates e.g. in between the lip and the dental soft tissue, in the dental pocket, or any other area in the oral cavity where the device is applicable, in order to elute NO at the area of interest when soluble film gets in contact with the moisture in the oral cavity, or between the inside of the cheek and the gum, in one direction or both.

In another embodiment of the present invention the nanoparticles, or microspheres, of the polymers in the present invention, may be encapsulated in a material that breaks upon the stress from chewing or brushing the teeth. Then said nanoparticles, or microspheres, may be integrated in chewing gum or toothpaste. This kind of chewing gum or toothpaste may then be used to prevent or treat disorders in the oral cavity, such as infections, cancer, or paradontosis, or to promote osteosynthesis and soft tissue healing post dental implant. The materials used to encapsulate these nanoparticles, or microspheres, may be chosen from the group comprising polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. This embodiment has the advantages that it is easy to apply, the treatment effect covers the whole oral cavity, and it is easy to manufacture.

In the context of the present invention the term “encapsulating” is intended to be interpreted as fixating the nitric oxide eluting polymer in a three dimensional matrix such as a foam, a film, a nonwoven mat of nanofibers or fibers, other materials with the capability to fixate the NO eluting polymer, or enclosing the nitric oxide eluting polymer in any suitable material.

In still another embodiment of the present invention the nanoparticles, or microspheres, may combined with a suitable mouthwash, such as chlorine dioxide (ClO₂), according to FIG. 6. When the mouthwash is used in the oral cavity, the nanoparticles, or microspheres, break and NO is released. Of course, chlorine dioxide may be combined with all the embodiments according to the present invention, such as the patch. This offers the advantage of further promoting the anti-bacterial effect of NO.

In yet another embodiment of the present invention the NO-eluting device is acting as a booster for drug eluting patches, e.g. pharmaceuticals, vitamins, nicotin, nitroglycerin etc. This embodiment presents a device with the advantage of combining two therapeutic treatments, of significant value, in one treatment. A synergetic effect may be that NO that is eluted from the device has a vasodilatory effect on the region where the device having the combination compound actuates. Vasodilated tissue is more susceptible to certain medications and thus more easily treated by the medical preparations and still NO has in addition to that the anti-inflammatory, anti-bacterial etc. effect. Hence, an unexpected surprisingly effective treatment is provided.

In still another embodiment the nitric oxide eluting polymer, such as powder, nanoparticles or microspheres, can be incorporated in foam. The foam may have an open cell structure, which facilitates the transport of the proton donor to the nitric oxide eluting polymer. The foam can be of any suitable polymer such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

In another embodiment the device is in the form of a cream, a gel or a combination of the two. This embodiment has the advantage of being able to penetrate pockets and corners, e.g. in the gum or skin for closer elution of NO on the area to be treated.

In another embodiment the device is in the form of a cream, a gel or a combination of the two. Since the nitric oxide eluting polymer is activated by proton donors the nitric oxide eluting polymer has to be separate from the proton donor until one wants to initiate the elution of nitric oxide, i.e. use the device. One way to accomplish this is to have a syringe with two separate containers. In one container you have a proton donor-based gel and in the other a non proton donor-based gel, comprising the nitric oxide eluting polymer. Upon using the device the two gels are squeezed from the syringe and mixed together, the proton donor in the first gel comes in contact with the nitric oxide eluting polymer in the second gel and the elution of nitric oxide starts.

In still another embodiment of the present invention dental implants, such as screws of titanium, and other biodegradable or biocompatible plates, may be integrated with the fibers, nanoparticles, or microspheres according to the present invention, e.g. by coating the devices. A very convenient way for coating is offered by electro-spinning or gas-jet spinning of NO eluting polymers onto the surface of the devices. This embodiment decreases the risk of infections during surgical procedures in the oral cavity.

The device may include materials such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. The NO-eluting polymer may be integrated in, spun together with, or spun on top of, any of these materials in all of the embodiments of the present invention.

Three important factors in controlling and regulating the elution of nitric oxide from a nitric oxide eluting polymer are how quickly a proton donor comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

In one embodiment of the present invention a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of an hydrophilic polymer is polyethylene oxide, and one example of an hydrophobic polymer is polystyrene. These carrier polymers may be mixed with the nitric oxide eluting polymer and then electrospun to suitable fibers. The skilled person in the art knows which other polymers may be used for similar purposes. FIG. 7 illustrates two elution profiles (NO concentration vs. time) for two different polymer mixtures; a nitric oxide eluting polymer mixed with a hydrophilic carrier polymer in an acidic environment (A), and a nitric oxide eluting polymer mixed with a hydrophobic carrier polymer in a neutral environment (B).

In one embodiment this carrier polymer is substituted by another material with hydrophobic or hydrophilic properties. Therefore, the term “carrier material” in the present context should be interpreted to include carrier polymers and other materials with hydrophilic or hydrophobic properties.

In another embodiment of the present invention the elution of nitric oxide from a nitric oxide eluting polymer, such as L-PEI-NO, is influenced by the presence of protons. This means that a more acidic environment provides a quicker elution of nitric oxide. By activating the nitric oxide eluting polymer, or mixture of nitric oxide eluting polymer and carrier material, with an acidic fluid, such as an ascorbic acid solution, the elution of nitric oxide may be accelerated.

The carrier polymers and carrier materials mentioned above may affect other characteristics than the regulation of nitric oxide elution. An example of such characteristic is mechanical strength.

In respect of the carrier polymers or carrier materials, the NO-eluting polymer may be integrated in, spun together with, or spun on top of, any of these materials in all of the embodiments of the present invention. This spinning includes electrospinning, air spinning, wet spinning, dry spinning, melt spinning, gel spinning. In this way, one may manufacture fibers of a polymer mixture, comprising a nitric oxide eluting polymer and a carrier polymer, or a carrier material, with predefined nitric oxide eluting characteristics. These characteristics may be tailor made for different elution profiles in different applications.

The device elutes nitric oxide (NO) from said eluting polymer in a therapeutic dose, such as between 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 1 to 1000 ppm.

In the embodiments of the present invention it may be suitable to control or regulate the time span of NO release from the device according to the invention. This may be accomplished by integrating other polymers or materials in said device. These polymers or materials may be chosen from any suitable material or polymer, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

The sizes of the devices according to the present invention may of course vary widely within in the parameters conveniently used in the oral cavity, but the size is typically 7 to 15 mm×20 to 40 mm, preferably 9 to 13 mm×25 to 35 mm, such as 10 mm×30 mm.

The NO-eluting polymers in the devices according to the present invention may be combined with silver, such as hydroactivated silver. The integration of silver in the devices according to the present invention gives the healing process an extra boost. Preferably the silver is releasable from the devices in the form of silver ions. The integration of silver in the device may present several advantages. One example of such an advantage is that the silver may keep the device in itself free from bacteria or viruses, while the nitric oxide eluting polymer elutes the therapeutic dosage of nitric oxide to the target site.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbor atom, such as a carbon atom, to the nitrogen atom, with higher electronegativity than nitrogen (N), it is very difficult to load the polymer with nitric oxide. On the other hand, if there is a electropositive ligand close to the secondary amine, such as on a neighbor atom, such as a carbon atom, to the nitrogen atom, the electronegativity of the amine will increase and thereby increase the possibility to load the nitric oxide elution polymer with nitric oxide.

In an embodiment of the present invention the nitric oxide polymer may be stabilized with a salt. Since the nitric oxide eluting group, such as a diazeniumdiolate group, usually is negative, a positive counter ion, such as a cation, may be used to stabilize the nitric oxide eluting group. This cation may for example be selected from the group comprising any cation from group 1 or group 2 in the periodic table, such as Na⁺, K⁺, Li⁺, Be²⁺, Ca²⁺, Mg²⁺, Ba²⁺, and/or Sr²⁺. Different salts of the same nitric oxide eluting polymer have different properties. In this way a suitable salt (or cation) may be selected for different purposes. Examples of cationic stabilized polymers are L-PEI-NO-Na, i.e. L-PEI diazeniumdiolate stabilized with sodium, and L-PEI-NO-Ca, i.e. L-PEI diazeniumdiolate stabilized with calcium.

Another embodiment of the present invention comprises mixing the nitric oxide eluting polymer, or a mixture of the nitric oxide eluting polymer and a carrier material, with an absorbent agent. This embodiment provides the advantage of an accelerated elution of nitric oxide since the polymer, or polymer mixture, via the absorbent agent, may take up the activating fluid, such as water or body fluid, much faster. In one example 80% (w/w) absorbent agent is mixed with the nitric oxide eluting polymer, or mixture of nitric oxide eluting polymer and carrier material, and in another embodiment 10 to 50% (w/w) absorbent agent is mixed with the nitric oxide eluting polymer, or mixture of nitric oxide eluting polymer and carrier material.

Since the elution of nitric oxide is activated by a proton donor, such as the water in the oral cavity, it may be an advantage to keep the nitric oxide eluting polymer, or mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. Therefore, in still another embodiment of the present invention, the elution of nitric oxide may be regulated by adding an absorbent agent. The absorbent agent absorbs the proton donor, such as water, and keeps the proton donor in close contact with the nitric oxide eluting polymer during prolonged periods of time. Said absorbent agent may be selected from the group comprising polyacrylates, polyethylene oxide, carboxymethylcellulose, and microcrystalline cellulose, cotton, and starch. This absorbent agent may also be used as a filling agent. In this case said filling agent may give the nitric oxide eluting polymer, or mixture of said nitric oxide eluting polymer and a carrier material, a desired texture.

The device may be manufactured by, for example electro spinning of L-PEI. L-PEI is the charged at a characteristic voltage, and a fine jet of L-PEI releases as a bundle of L-PEI polymer fibers. This jet of polymer fibers may be directed to a surface to be treated. The surface to be treated may for example be any suitable material in respect of a device. The electro spun fibers of L-PEI then attach on said material and form a coating/layer of L-PEI on the device according to the invention.

It is of course possible to electro spin the other NO-eluting polymers, according to above, on the device according to the invention while still being inside the scope of the present invention.

Other manufacturing methods, such as wet spinning, dry spinning, melt spinning, and gel spinning, are also within the scope of the present invention.

In one embodiment the NO-eluting polymers according to the present invention are electro spun in such way that pure NO-eluting polymer fibers may be obtained.

Gas stream spinning, air spinning, wet spinning, dry spinning, melt spinning, and gel spinning, of said NO-eluting polymers onto the device is also within the scope of the present invention.

The manufacturing process according to the present invention presents the advantages of large contact surface of the NO-eluting polymer fibers with the area to be treated, effective use of NO-eluting polymer, and a cost effective way of producing the device.

The device according to the invention may of course be used in any post surgery treatment to prevent, treat, and/or alleviate any kind of infection or inflammation. Especially to prevent disorders post surgery in the oral cavity. The effects of NO are provided in a convenient way by the device of the invention. Such effects are for instance, as mentioned above, anti-inflammatory, anti-pathogenic, especially anti-viral and anti-bacterial. Furthermore the anti-cancerous effect of NO may be taken advantage of, e.g. for bone cancer treatment of the jaw.

Hereinafter some potential uses of the present invention are described:

A method of therapeutically treating disorders in the oral cavity, comprising introducing a stick or pin having releasably attached thereto a device according to the present invention into the oral cavity of a patient, releasing the device, in the oral cavity of the patient, from the stick or pin, thereby contacting an area of treatment in the oral cavity, such that a therapeutic dose of nitric oxide is eluted from said nitric oxide eluting polymer to said area.

The method according to the above, wherein said area of treatment is an infected area in the oral cavity or an area where infection is to be prevented.

The method according to the above, wherein said treatment area is a post-operative dental surgery area.

The method according to the above, wherein said treatment area is a tumor area in the oral cavity.

Use of nitric oxide (NO) in a therapeutic dose for the treatment of disorders in the oral cavity, wherein said use for instance is the treatment or prevention of paradontosis.

The invention can be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

What is claimed is:

1. A non-implantable device configured to topically therapeutically treat and/or prevent disorders in the oral cavity, wherein
said device comprises a nitric oxide (NO) eluting polymer configured to elute a therapeutic dosage of nitric oxide (NO) when used for said treatment and/or prevention of a target site in the oral cavity, and wherein said device is configured for exposure of said target site to said nitric oxide when said polymer elutes nitric oxide (NO), and wherein
said nitric oxide (NO) eluting polymer is integrated with a carrier material, wherein said carrier material regulates the elution of said therapeutic dosage of nitric oxide (NO),
wherein said device is a pad/patch, a condom/sheath, a soluble film, a sponge, a cream, or a gel, and is adapted to be applied to the oral cavity, wherein
said device is activatable immediately prior to introduction into said oral cavity or in said oral cavity for release of said nitric oxide (NO) from said nitric oxide (NO) eluting polymer, and independent of humidity in said oral cavity, by a proton donor provided separately from said nitric oxide (NO) eluting polymer.

2. The device according to claim 1, wherein said elution of nitric oxide (NO) from said device is substantially directed towards said target site for said exposure.

3. The device according to claim 1, further comprising a first membrane, which is permeable to nitric oxide on a first side of the device, wherein said first side is oriented towards said treatment site, and a second membrane which has low permeability or substantially no permeability to nitric oxide on a second side of said device, wherein said second side is oriented away from said treatment site, such that said substantial direction of nitric oxide (NO) from said device is provided as the elution of nitric oxide from said device and is substantially prevented from said second side.

4. The device according to claim 1, wherein said nitric oxide (NO) eluting polymer comprises diazeniumdiolate groups, S-nitrosylated groups, O-nitrosylated groups, or any combination thereof.

5. The device according to claim 1, wherein said nitric oxide (NO) eluting polymer is L-PEI (linear polyethyleneimine).

6. The device according to claim 1, wherein said nitric oxide eluting polymer is selected from the group consisting of amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, and any combination thereof, wherein said polymer is grafted to an inert backbone selected from the group consisting of a polysaccharide backbone and a cellulosic backbone.

7. The device according to claim 1, wherein said device is configured to disintegrate in the oral cavity when subjected to moisture or water.

8. The device according to claim 1, wherein said NO-eluting polymer is combined with silver.

9. The device according to claim 1, wherein said carrier material is selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, latex, and any combination thereof.

10. The device according to claim 1, wherein said nitric oxide eluting polymer is in the form of nanoparticles or microspheres.

11. The device according to claim 10, wherein said nanoparticles or microspheres are encapsulated in a material selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, latex, and any combination thereof.

12. The device according to claim 10, wherein said nanoparticles or microspheres are integrated into a toothpaste.

13. The device according to claim 1, wherein said nitric oxide eluting polymer comprises a secondary amine in the backbone or a secondary pendant amine.

14. The device according to claim 13, wherein a positive ligand is located on a neighbor atom to the secondary amine.

15. The device according to claim 1, further comprising an absorbent agent.

16. The device according to claim 15, wherein said absorbent agent is selected from the group consisting of polyacrylate, polyethylene oxide, Carboxy Methyl Cellulose (CMC), microcrystalline cellulose, cotton, starch, and any combination thereof.

17. The device according to claim 1, further comprising a cation, wherein said cation stabilizes the nitric oxide eluting polymer.

18. The device according to claim 17, wherein said cation is selected from the group consisting of Na⁺, K⁺, Li⁺, Be²⁺, Ca²⁺, Mg²⁺, Ba²⁺, Sr²⁺, and any combination thereof.

19. The device according to claim 1, wherein the nitric oxide eluting polymer is in the form of a powder, nanoparticles or microspheres, which is incorporated into a foam.

20. The device according to claim 19, wherein the foam has an open cell structure configured to facilitate transport of said proton donor to the nitric oxide eluting polymer.

21. The device according to claim 20, wherein the foam comprises a polymer selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, latex and any combination thereof.

22. The device according to claim 1, wherein the device is a syringe-type device having two separate containers, wherein a first container contains a proton donor-based NO release activation agent, and a second container contains a non proton donor-based gel, comprising the nitric oxide eluting polymer, wherein the syringe-type device is configured to provide admixing upon administration.

23. The device according to claim 1, wherein the device is further activatable in said oral cavity for release of said nitric oxide (NO) from said nitric oxide (NO) eluting polymer by humidity in said oral cavity.

24. A manufacturing process for a device configured to therapeutically treat and/or prevent disorders in the oral cavity according to claim 1, comprising:
selecting a nitric oxide (NO) eluting polymer configured to elute a therapeutic dosage of nitric oxide (NO) in the oral cavity when used for said therapeutic treatment and/or prevention;
selecting a carrier material configured to regulate the elution of said therapeutic dosage of nitric oxide (NO);
incorporating the NO-eluting polymer with said carrier material into an nitric oxide (NO) eluting material, wherein said carrier material regulates the elution of said therapeutic dosage of nitric oxide (NO); and
deploying said nitric oxide eluting material to form at least a part of said device, such that said device is configured to expose a therapeutic target site in the oral cavity to said nitric oxide when said NO-eluting polymer elutes nitric oxide (NO); and
providing said device as a pad/patch, a condom/sheath, a soluble film, a sponge, a cream, or a gel, wherein said device is adapted to be applied in the oral cavity, wherein said device is activatable immediately prior to an introduction into said oral cavity, or in said oral cavity, for release of said nitric oxide (NO) from said nitric oxide (NO) eluting polymer, and independent of humidity in said oral cavity, by a proton donor provided separately from said nitric oxide (NO) eluting polymer.

25. The manufacturing process according to claim 24, further comprising
applying a material that has low permeability or substantially no permeability to nitric oxide (NO) on a side of device that is intended to be oriented away from said therapeutic target site, wherein elution of nitric oxide is substantially directed towards said therapeutic target site.

26. The manufacturing process according to claim 24, wherein said deploying comprises electro spinning, air spinning, gas spinning, wet spinning, dry spinning, melt spinning, or gel spinning of said NO-eluting polymer.

27. The manufacturing process according to claim 24, wherein said selecting said nitric oxide (NO) eluting polymer comprises selecting a plurality of nitric oxide (NO) eluting polymeric particles selected from the group consisting of nanofibers, nanoparticles and microspheres.

28. The manufacturing process according to claim 24, wherein said incorporating said NO-eluting polymer with said carrier material comprises integrating said NO-eluting polymer in said carrier material, spinning said NO-eluting polymer together with said carrier material, or spinning said NO-eluting polymer on top of said carrier material.

29. The manufacturing process according to claim 24, further comprising integrating silver into said device.

30. A method of therapeutically treating a disorder in the oral cavity, comprising:
introducing a stick or pin having releasably attached thereto a device according to claim 1 into the oral cavity of a patient; and
releasing the device in said oral cavity from the stick or pin, thereby contacting an area of treatment in the oral cavity, such that a therapeutic dose of nitric oxide is eluted from said nitric oxide eluting polymer towards said area.

31. The method according to claim 30, wherein said area of treatment is an infected area or an area where infection is to be prevented.

32. The method according to claim 30, wherein said treatment area is a post-operative dental surgery area.

33. The method according to claim 30, wherein said treatment area is a tumor area.

34. The method according to claim 30, wherein said treatment area is an area having paradontosis.
